(19) United States
(12) Patent Application Publication
(10) Pub. No.: US 2026/0345716 A1
(43) Pub. Date: Nov. 5, 2026

(54) WITHANIA SOMNIFERA COMPOSITION FOR COGNITIVE HEALTH

(71) Applicant: OMNIACTIVE HEALTH TECHNOLOGIES LIMITED, Mumbai (IN)

(72) Inventors: Muralidhara PADIGARU, Thane (IN); Abhijeet MORDE, Thane (IN); Sumit PATIL, Thane (IN); Ravindra NAYAKWADI, Thane (IN); Ninad PURANIK, Thane (IN); Nupur NAGAVEKAR, Thane (IN); Bhushan CHAUDHARI, Thane (IN)

(21) Appl. No.: 19/104,986

(22) PCT Filed: Aug. 21, 2024
(86) PCT No.: PCT/IN2024/051524

§ 371 Date: Feb. 19, 2025

(30) Foreign Application Priority Data
Aug. 24, 2023 (IN) 202321029399

(51) Int. Cl. A61K 36/81; A61K 9/00; A61K 9/10; A61K 9/16; A61K 31/585; A61K 31/7048; A61K 47/14; A61K 47/24; A61K 47/44; A61P 25/20; A61P 25/22

(52) U.S. Cl. CPC A61K 36/81; A61K 9/0053; A61K 9/10; A61K 9/1611; A61K 9/1617; A61K 9/1652; A61K 9/1694; A61K 31/585; A61K 31/7048; A61K 47/14; A61K 47/24; A61K 47/44; A61P 25/20; A61P 25/22; A61K 2236/333; A61K 2236/51; A61K 2236/53

(57) ABSTRACT

The present invention relates to an oral composition of Withania extract as or its salts and/or in combination with total Withanolides comprising Withanolide Glycosides (Withanoside IV and Withanoside V/VI) and Withanolide aglycones (Withanolide A, Withanolide B, Withaferin-A and 12-deoxy withastramonolide) and/or its salts. The present invention is also related to the process of preparation of the oral composition Withania somnifera and at least one pharmaceutically and/or nutraceutically acceptable excipients for prevention, improvement, and maintenance of cognitive health to a subject in need thereof. The invention also discloses the process for preparing extracts from plant parts of Withania somnifera.

The composition of the present invention is available in dosage forms like powder, beadlets, granules, capsules, tablets, oil suspension, films or any other suitable oral or topical dosage forms used for prevention, improvement and maintenance of cognitive health.

### FIELD OF THE INVENTION

The present invention relates to the oral composition of enriched Withania somnifera (commonly called Ashwagandha) extract comprising withanolide glycosides and withanolide aglycones. Also, a present invention relates to the use of Withania somnifera extract in the form of composition for prevention, improvement, and maintenance of cognitive health, more particularly to stress, reducing cortisol and anxiety and improving sleep to a subject in need thereof. Further, the invention provides a composition comprising the enriched and/or standardized Withania somnifera (Ashwagandha) extract with at least one pharmaceutically and/or nutraceutical-acceptable excipient, resulting in enhanced bioavailability.

Furthermore, the invention relates to the oral composition of Withania somnifera wherein the enriched extract comprising Withania somnifera (Ashwagandha) extract as a whole, or its salts and/or in combination with enriched and/or standardized constituents in a suitable selective ratio of withanolides comprises of withanolide glycosides selected from withanoside IV, withanoside V/VI, and enriched withanolide aglycones selected from withanolide A, withanolide B, withaferin-A and 12-deoxywithastramonolide and/or its salts and derivatives

### BACKGROUND OF THE INVENTION

Cognitive health is an integral part of health; it is more than the absence of cognitive illnesses. It is the foundation for the well-being and effective functioning of individuals. It includes cognitive well-being, prevention of cognitive disorders, treatment, and rehabilitation. Particularly, cognitive health means having a healthy brain and healthy body that work in tandem to achieve overall wellness.

Cognitive health refers to a broad range of cognitive processes, including managing stress, reducing cortisol, managing anxiety and improving sleep quality. Recent studies indicate that several negative lifestyle factors can contribute to the development of cognitive impairment.

WHO estimates that the burden of cognitive health problems in India is 2443 disability-adjusted life years (DALYs) per 100 00 population; the age-adjusted suicide rate per 100 000 population is 21.1. Worldwide, the economic loss due to cognitive health conditions, between 2012-2030, is estimated at USD 1.03 trillion.

In 2019, 1 in every 8 people, or 970 million people around the world were living with a cognitive disorder, with anxiety and depressive disorders the most common. In 2020, the number of people living with anxiety and depressive disorders rose significantly because of the COVID-19 pandemic. Depression and anxiety went up by more than 25% in the first year of the pandemic alone. Initial estimates show a 26% and 28% increase respectively for anxiety and major depressive disorders in just one year. While effective prevention and treatment options exist, most people with cognitive disorders do not have access to effective care. Many people also experience stigma, discrimination and violations of human rights.

United States Patent Publication No. US 20210369804 A1 discloses an enriched Withania somnifera extract composition comprising 3-15% of withanolide glycosides, 2.5-8% of withanolide aglycones, and 0.01-0.7% of withaferin-A and its process of extraction. The application also covers the use of enriched extraction for improving testosterone levels, energy levels, sustained energy, vigor, stamina, muscle mass, and muscle strength, reducing cortisol and stress.

United States Patent Publication No. US 20220409635 A1 discloses Withania somnifera extract composition comprising at least one of withaferin A, withanoside IV, withanoside V, withanoside VI, withanolide A, withanolide B, withaferin, anahygrine, 12-deoxywithastromonolide, sitoindoside X, sitoindoside IX, cuscohygrine, isopelletierine, bracteosin A, bracteosin B, or bracteosin C, or a salt or solvate of any one thereof. Related compositions, methods, and processes are also provided therein.

International Publication No. WO 2022150918 A1, discloses compositions with enriched amounts of Withanolides consisting entirely of natural extracts of Ashwagandha (Withania somnifera), which have been supplemented with additional amounts of withanolide A, withanolide B, withanolide D, withaferin A, withanoside IV, and/or withanoside V & methods of use thereof. The '918 application also discloses the methods for modulating GPX1, Nrf2, H01, mTOR, TERT, -Catenin, and/or NF-_{κ}B using such Withanolide-enriched compositions.

U.S. Pat. No. 8,501,186 B2 discloses vaccine preparation with adjuvant comprising Withania somnifera fraction rich in withanolides. In particular, Withania somnifera fraction rich in withanolides; said fraction comprising withanolide A of about 0.5-1%; withaferin A of about 0.1-0.6%; withanolide B of about 0.01-0.1%; withanoside IV of about 0.8-1.2%; withanoside V of about 0.5-0.8%; and 12-deoxywithastramonolide of about 0.8-1.2%; wherein the ratio of withaferin A to withanolide A is in the range of about 1:2 to about 1:5.

To the best of the applicant's knowledge, none of the prior art, not limited to the above listed arts, anticipate the present invention. The present invention is primarily related to the Withania somnifera composition comprising enriched withanolide glycosides selected from withanoside IV, withanoside V/VI, and enriched withanolide aglycones selected from withanolide A, withanolide B, withaferin-A and 12-deoxywithastramonolide and/or its salts in specific proportion.

Although many health benefits of the Withania somnifera (Ashwagandha) extract are known, none of the references focuses on their role in the prevention, improvement, and maintenance of cognitive health. Medical advances have improved treatments of numerous diseases, but the cognitive implications have not been sufficiently addressed.

Thus, there is a need to have an effective solution for the prevention, improvement, and maintenance of cognitive health, particularly through dietary supplementation, which is convenient for the person in need.

### DESCRIPTION OF FIGURES

FIG. 1: Chemical structure of withanolide glycosides

FIG. 2: Chemical structure of withanolide aglycones.

FIG. 3: Screening of Subjects.

FIG. 4: Summary of total withanolide plasma concentration for Product A, Product B, Product C, and Product D.

FIG. 5: Effects of different Withania somnifera extract doses on entries to target quadrant (A), probe trial (B), and spatial learning memory acquisition phase (SMAP) (C) in sleep-deprived rats.

FIG. 6: Effects of different Withania somnifera extract doses on Serum MDA (A), Liver MDA (B), Brain MDA (C), Brain SOD (D), Brain CAT (E), Brain GSH-Px (F), TAC (G) in sleep-deprived rats.

FIG. 7: Effects of different Withania somnifera extract doses on Corticosterone (A) CRH (B) ACTH (C) Serotonin (D) Dopamine (E) in sleep-deprived rats.

FIG. 8: Effects of different Withania somnifera extract doses on brain tissue NCAM (A), ICAM-1 (B), BDNF (C), and NGF (D) protein levels in sleep-deprived rats.

FIG. 9: Effects of different Withania somnifera extract doses on brain tissue GABAAR2 (A), GABABR1 (B), GABABR2 (C), GAP-43 (D), and 5-HT1A (E) protein levels in sleep-deprived rats.

### OBJECTIVES OF THE INVENTION

The primary objective of the present invention is to provide the oral composition of Withania somnifera (Ashwagandha) comprising enriched and/or standardized Withania somnifera (Ashwagandha) extract as whole or its salts and/or in combination with enriched active constituents in a selective ratio of withanolides comprises of withanolide glycosides selected from withanoside IV, withanoside V/VI, and enriched withanolide aglycones selected from withanolide A, withanolide B, withaferin-A and 12-deoxywithastramonolide and/or its salts and derivatives.

Another objective of the present invention is an oral composition of enriched Withania somnifera (Ashwagandha) extract comprising withanolides which comprise withanolide glycosides selected from withanoside IV and withanoside V/VI, and withanolide aglycones selected from withanolide A, withanolide B, withaferin-A and 12-deoxywithastramonolide) and/or its salts and at least one pharmaceutically and/or nutraceutically acceptable excipients and relates to use for the prevention, improvement, and maintenance of cognitive health, more particularly to improving stress, reducing cortisol and anxiety and improving sleep to a subject in need thereof.

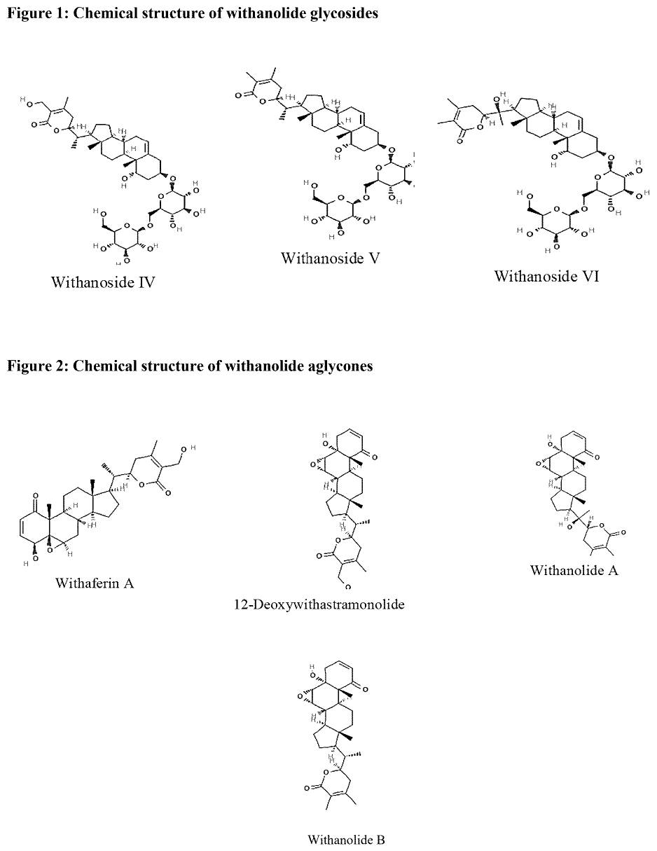

Furthermore, the objective of the invention is to provide a process for extraction with enrichment of Withania somnifera (Ashwagandha) extract as whole or its salts and/or in combination with suitable active constituents comprises withanolides, in suitable selective ratio and/or in preferred percentages, comprises of withanolide glycosides selected from withanoside IV and withanoside V/VI (FIG. 1), and withanolide aglycones selected from withanolide A, withanolide B, withaferin-A and 12-deoxywithastramonolide (FIG. 2) and/or its salts and at least one pharmaceutically and/or nutraceutically-accepted excipients, resulting in enhanced bioavailability.

One more objective of the invention is to provide standardized and/or enriched the active constituents in the extract composition of Withania somnifera (Ashwagandha) in suitable ratios and/or percentages to exert the synergetic effect.

One more objective of the present invention of oral Withania somnifera compositions is to provide a suitable formulation and process that results in enhanced bioavailability measured by PK studies using excipients selected from the group of, but not limited to, bioavailability enhancers, PGP inhibitors, pH modifiers, emulsifiers, carriers, anticaking agents, coating agents and oils, or the combination thereof.

Also, the objective of the present invention is to provide the oral composition of enriched Withania somnifera extract of withanolides comprises of withanolide glycosides selected from withanoside IV and withanoside V/VI, and withanolide aglycones selected from withanolide A, withanolide B, withaferin-A and 12-deoxywithastramonolide which helps to reduce stress, reduce cortisol, manage anxiety and improve sleep quality Further objective of the present invention is to provide an oral composition of enriched Withania somnifera (Ashwagandha) extract and active constituents comprises of withanolides consisting of withanolide glycosides selected from withanoside IV and withanoside V/VI, and withanolide aglycones selected from withanolide A, withanolide B, withaferin-A and 12-deoxywithastramonolide) and/or its salts in a suitable ratio.

One more objective of the invention is to provide a process for the preparation of enriched Withania somnifera (Ashwagandha) extract and composition as whole or its salts and/or in combination with active constituents of withanolides comprising of withanolide glycosides selected from withanoside IV and withanoside V/VI, and withanolide aglycones selected from withanolide A, withanolide B, withaferin-A and 12-deoxywithastramonolide and/or its salts is extracted from the stems and roots of the plant.

Further objective of the present invention is to provide an oral enriched compositions of Withania somnifera (Ashwagandha) extract is formulated in different forms like orally administrable solid, semisolid, and liquid forms, selected from, but not limited to dosages such as powders, granules, beadlets, caplets, tablets, capsules, soft gel capsules, solution, emulsions, suspensions, oil suspensions, dispersions and the like.

### DETAILED DESCRIPTION OF THE INVENTION

Ashwagandha is one of the most valuable medicinal plants, forming the active component or whole of the phytopharmaceutical products for the prevention and cure of a variety of human ailments and diseases. Ayurvedic, Unani and Sidha medicinal products based on the plant currently run above one hundred, and it is rapidly expanding worldwide. Withanolide glycosides and withanolide aglycones of Withania somnifera, also known as Ashwagandha or winter cherry, is an evergreen shrub belonging to the family Solanaceae or nightshade family and the genus withania.

The present invention is also related to an oral composition of Withania somnifera enriched and/or standardized extract wherein Withania somnifera extract comprises withanolides comprising of withanolide glycosides and withanolide aglycones.

Wherein the enriched can considered to be made rich or richer, especially by the addition or increase of some desirable quality, attribute, or ingredient. Also, standardized extract means a standardized extract is one that is manufactured to contain a consistent level of one or more phytochemical constituents which are derived from the original starting material. The enrichment and/or standardization of extract is subject to the requirement to arrive at a final required composition.

In another embodiment, the present invention provides an oral composition of Withania somnifera extract, wherein the extract comprises withanolide glycosides selected from withanoside IV and withanoside V/VI and withanolide aglycones selected from withanolide A, withanolide B, withaferin-A, and 12-deoxywithastramonolide in a sitable ratios.

The inventors of the invention conducted rigorous experimentation to arrive at a selective range of constituents as total withanolides about to 1% to 40% and total saponin of about 20% to 60% in the Withania somnifera extract; particularly 3% to 30% in the Withania somnifera extract; more particularly total withanolides of 5% to 25% in the Withania somnifera extract.

In one embodiment, the total withanolides in Withania somnifera extract comprise withanolide glycosides in the range of 3% to 9.7% of total withanolides and withanolide aglycones in the range of 8% to 16.6% of total withanolide.

In another embodiment, withanolide glycosides are selected from 0.54% to 1.94% of withanoside IV and 0.48% to 1.94% of withanoside V/VI and withanolide aglycones are selected from 1% to 3.82% of withanolide A, 0.01% to 0.16% of withanolide B, 2.24% to 5% of withaferin-A and 0.96% to 2.33% of 12-deoxywithastramonolide. The percentage of all the withanolides is measured by the HPLC method provided in the US Pharmacopeia as USP 43-NF38-4768.

In one aspect the present invention provides oral composition of Withania somnifera extract with total withanolides of 5% to 25% wherein total withanolides comprises 3% to 9.7% of withanolide glycosides selected from 0.54% to 1.94% of withanoside IV and 0.48% to 1.94% of withanoside V/VI and 8% to 16.6% of withanolide aglycones selected from 1% to 3.82% of withanolide A, 0.01% to 0.16% of withanolide B, 2.24% to 5% of withaferin-A and 0.96% to 2.33% of 12-deoxywithastramonolide.

In another aspect the present invention provides the composition of Withania somnifera extract for prevention, improvement, and maintenance of cognitive health. More specifically, the present invention is to provide the oral Withania somnifera composition, which improves the level of stress by reducing cortisol, managing anxiety and improving sleep quality. Based on initial screening of the oral composition, the inventors of this instant invention surprisingly observed an enhancement in enhanced efficacy due to more in bioavailability of the selective range of active constituents of Withania somnifera as defined in the invention.

The term “oral composition or oral Withania somnifera composition” as used throughout the specification refer to a composition having Withania somnifera (Ashwagandha) extract as a whole and/or selectively oral composition of enriched Withania somnifera (Ashwagandha) extract and active constituents comprises of withanolides consisting of withanolide glycosides selected from withanoside IV and withanoside V/VI, and withanolide aglycones selected from withanolide A, withanolide B, withaferin-A and 12-deoxywithastramonolide) and/or its salts with one or more pharmaceutically and/or nutraceutically acceptable excipients.

The term “Withania somnifera extract” as used throughout the specification can also be interpreted to be within the scope of, which in general includes Withania somnifera extract as whole or its salts and/or in combination with of withanolides comprising of withanolide glycosides selected from withanoside IV and withanoside V/VI and withanolide aglycones selected from withanolide A, withanolide B, withaferin-A, and 12-deoxywithastramonolide and/or its salts in a selective ratio.

The term “enrichment of Withania somnifera extract” as used throughout the specification can be interpreted to be within the scope of the external addition of active constituents in the Withania somnifera extract to meet the selective ratio of all active constituents.

According to an embodiment of the present invention, oral compositions of the invention are comprised of the Withania somnifera extract, comprising Withania somnifera extract as a whole, oral composition of withanolides comprising of withanolide glycosides selected from withanoside IV and withanoside V/VI and withanolide aglycones selected from withanolide A, withanolide B, withaferin-A, and 12-deoxywithastramonolide and/or its salts and one or more pharmaceutically and/or nutraceutically acceptable excipients.

One of the embodiments of the present invention provides a process for preparing extracts of the invention from plant parts of Withania somnifera wherein the process comprises:
(i) obtaining a required material from one or more parts of the Withania somnifera plant;
(ii) obtaining an extract from the plant material by soaking it into an aqueous solvent and/or an organic solvent, or a combination thereof, for a suitable period and temperature to extract out the required constituents;
(iii) stirring the soaked plant material at a suitable temperature of about 50 °C to about 65 °C for a specified period;
(iv) optionally adjusting the pH using a pH modifier, allowing it tlo settle at room temperature and filtering; followed by washing with water to remove the plant material;
(v) drying and collecting the powder.

According to a further embodiment, the aqueous solvent can be selected from the group consisting of water, phosphoric acid, hydrochloric acid and combination thereof. Also, an organic solvent can be selected from the group consisting of methanol, ethanol, propanol, butanol ethyl acetate, acetone, hexene, chloroform, and a combination thereof at a suitable temperature.

In one embodiment, the present invention provides the process for preparing extracts of Withania somnifera wherein the process comprises:
(i) charging 350±50 Kg of Ashwagandha Root (Regular Grade)-cut in extractor;
(ii) preparing 50-55% ethanol solution by adding required quantity of water;
(iii) charging 50-55% ethanol solution with respect to 5 volumes to input reaction mixture in extractor;
(iv) applying hot steam to the extractor to achieve temperature of about 60 °C to about 65 °C;
(v) circulating 50-55% ethanol solution for extraction at temperature of about 60 °C to about 65 °C for 3 hours;
(vi) separating the first wash through decanter and transferring the liquid extract to concentrator or reactor for distillation;
(vii) repeating the steps (iii), (iv), (v) and (vi) for separating the second wash and third wash through decanter and transferring the liquid extract to concentrator or reactor for distillation;
(viii) collecting all the washes and charging them to concentrator or reactor for distillation; and
(ix) drying and collecting the powder

According to a further embodiment, the Withania somnifera extract comprises Withania somnifera and/or its salts not less than 5% w/w of the total extract.

According to a further embodiment, a Withania somnifera extract comprises Withania somnifera and/or its salts not less than 10% w/w of the total extract.

According to a further embodiment, a Withania somnifera extract comprises Withania somnifera and/or its salts not less than 15% w/w of the total extract.

According to a further embodiment, the Withania somnifera extract comprises withanolide glycosides and/or its salts not less than 2% w/w of total withanolide extract.

According to a further embodiment, the Withania somnifera extract contains withanolide aglycones and/or its salts not less than 5% w/w of the total extract.

Oral compositions described herein are comprised of enriched Withania somnifera extract as whole or its salts and/or in a combination of its active components to meet the selective ratio/percentage selected from total withanolides comprises withanolide glycosides (withanoside IV and withanoside V/VI) and withanolide aglycones (withanolide A, withanolide B, withaferin-A and 12-deoxywithastramonolide) and/or its salts and one or more of pharmaceutically and/or nutraceutically acceptable excipients.

More preferably, oral Withania somnifera compositions are formulated using excipients selected from the group comprising of, but not limited to, bioavailability enhancers, PGP inhibitors, pH modifiers, emulsifiers, carriers, anticaking agents, coating agents and oils, or the combination thereof.

According to a further embodiment, oral compositions described herein can be available in orally administrable solid, semisolid, and liquid forms, selected from, but not limited to, dosages such as, powders, granules, pellets, beadlets, caplets, tablets, capsules, soft gel capsules, solution, emulsions, suspensions, oil suspensions, dispersions and the like.

Oral compositions, as described herein are comprised of Withania somnifera extract as whole or its salts and/or in combination with total withanolides comprising withanolide glycosides (withanoside IV and withanoside V/VI) and withanolide aglycones (withanolide A, withanolide B, withaferin-A and 12-deoxywithastramonolide) and/or its salts with at least one excipient such as pharmaceutically and/or nutraceutically acceptable excipients which can be formulated as granules, powder and/or beadlets.

In one embodiment, the present invention provides a process for the preparation of an oral Withania somnifera composition in the form of powder wherein the process comprises—
(i) charging a solvent in a clean vessel;
(ii) adding bioenhancer, PGP inhibitor, pH modifier, and emulsifier under stirring to obtain a clear solution and heating the solution at temperature of about 50 °C to about 65 °C;
(iii) adding enriched Withania somnifera extract to the solution obtained in step (ii) under stirring and discontinuing the heating once a clear solution is observed;
(iv) adding carrier to a solution, obtained in step (iii) under stirring; and
(v) spray drying the solution obtained from step (iv) to get powder.

In another embodiment, the present invention provides a process for the preparation of oral Withania somnifera composition in the form of beadlets wherein the process comprises—
(i) preparing a solution of binder in a solvent;
(ii) adding enriched Withania somnifera extract composition, Anti-tacking agent and Diluent, and dry mix to form uniform blend
(iii) adding solutions prepared in step (i) in the dry blend of step (ii) to obtain the wet mass;
(iv) passing the wet mass through the extruder, followed by a spheronizer;
(v) drying and sifting the granules obtained in step (iv);
(vi) preparing coating solution using coating agent and solvent;
(vii) loading granules obtained in step (v) in fluidized bed coater and the solution obtained in step (vi); and
(viii) drying the granules obtained in step (vii).

In another aspect, the present invention provides a process for the preparation of an oral Withania somnifera composition in the form of oil suspension wherein the process comprises—
(i) adding bio-enhancers, PGP inhibitors, and emulsifiers in oils under stirring in a vessel and heating the mixture at temperature of about 50 °C to about 65 °C; and
(ii) adding enriched Withania somnifera extract to step (i) under stirring to obtain oil suspension.

In one aspect, the present invention provides a process for the preparation of oral Withania somnifera composition in the form of granules wherein the process comprises—
(i) preparing a solution of Binder, and bioenhancer separately in a solvent;
(ii) adding enriched Withania somnifera extract, Anti-tacking agent and Diluent in mixer to form a uniform blend;
(iii) adding solutions prepared in step (i) in the dry blend of step (ii) to obtain the wet granules; and
(iv) drying and sifting the granules obtained in step (iii).

In one more embodiment, the solvent used in preparation of oral composition is selected from the group comprising of, but not limited to, isopropyl alcohol, acetone, methanol, alcohol, ethyl acetate, ethanol, methylene dichloride, water, and a combination thereof.

In one more embodiment, the bioavailability enhancer or bioenhancer employed in the preparation of oral compositions is selected from the group comprising of, but not limited to, piperine, d-limonene, monoglycerides, and phospholipids such as lecithin/phosphatidyl choline, glyceryl monosterate botanical extracts and a combination thereof.

In one of the embodiments, the PGP inhibitor as used in preparation of the oral composition is selected from the group comprising of, but not limited to, piperine, Vitamin E TPGS (d-α-Tocopheryl polyethylene glycol 1000 succinate), alkaloids, polysorbates flavonoids, coumarins, resins, saponins and combination thereof.

In another aspect, the pH modifier as used in preparation of the oral composition is selected from the group comprising of, but not limited to sodium bicarbonate, citric acid, trisodium citrate, lactic acid, L-arginine, calcium carbonate, magnesium carbonate, and combination thereof.

In one aspect, the emulsifier as used in preparation of oral composition is selected from the group comprising of, but not limited to, stearic acid, phosphadylcholine, polysorbate, sugar alcohols, glycerol derivatives and combination thereof.

In one aspect, the polymer as used in preparation of oral composition is selected from the group comprising of, but not limited to, hydroxy propyl methyl cellulose, hydroxyl propyl cellulose, sodium carboxyl methyl cellulose, sodium alginates, carbomer and combination thereof.

In another embodiment, the carrier employed in preparation of oral compositions is selected from the group comprising of, but not limited to cellulose derivatives, like hydroxypropyl methyl cellulose, microcrystalline cellulose, polyacrylates, polyethylene glycols, povidones, starch and starch derivatives, gums, sugars, plant polysaccharides and combination thereof.

In another embodiment, the anticaking agent used in preparation of oral composition is selected from the group comprising of, but not limited to silicon dioxide, magnesium stearate, stearic acid, mannitol and combination thereof.

In one aspect, the coating agent employed in preparation of oral composition is selected from the group comprising of, but not limited to lipids, waxes, stearic acid, glyceryl distearate, shellac, zein, acrylate polymer, cellulose polymer and combination thereof.

In another aspect of the invention, carrier, oil and/or vehicle may be selected from the group comprising of, but not limited to, sunflower oil, coconut oil, corn oil, cottonseed oil, canola oil, olive oil, palm oil, peanut oil, safflower oil, sesame oil, soybean oil, rapeseed oil and medium-chain triglyceride (MCT) oil and the combination thereof.

In another embodiment, the present invention provides an oral composition of Withania somnifera extract comprising Withania somnifera extract as whole or its salts and/or in combination with total withanolides comprising withanolide glycosides selected from withanoside IV and withanoside V/VI and withanolide aglycones selected from withanolide A, withanolide B, withaferin-A and 12-deoxywithastramonolide and/or its salts is used for prevention, improvement, and maintenance of cognitive health, more specifically, which helps to reduce stress and cortisol, manage anxiety and improve sleep quality.

The present invention also provides the study to assess the efficacy of the oral composition of Withania somnifera extract composition comprising Withania somnifera extract as whole or its salts and/or in combination with total withanolides comprising withanolide glycosides (withanoside IV and withanoside V/VI) and withanolide aglycones (withanolide A, withanolide B, withaferin-A and 12-deoxywithastramonolide) and/or its salts prevention, improvement and maintenance of cognitive health. More particularly a Withania somnifera composition helps to reduce stress and cortisol, manage anxiety and improve sleep quality.

Source of the herb/plant parts used in the invention Withania somnifera (Ashwagandha) collected from a vendor from India—Dhanvantari Ayurveda, Ganesh Nagar, Near Khinvasara Patil, Chinchwad, Pune 411033, Maharashtra and Saraogi Agro Products LLP, Jaju Nagar, Neemuch, Madhya Pradesh 458441.

Those of ordinary skilled in the art will appreciate that changes could be made to the embodiments described above without departing from the broad inventive concept thereof. It is understood, therefore, that this invention is not limited to the embodiments or examples disclosed herein but is intended to cover modifications within the objectives and scope of the present invention as defined in the specification. The presented examples illustrate the invention, but they should not be considered to limit the scope of the invention in any way.

### Withania somnifera Extract Preparation

### Example 1

To Withania somnifera root powder (100 g) was added 50% ethanol (500 mL) and extracted at temperature of about 60-65 °C for 3 hours. The mixture was filtered through nutsche filter (2 microns). The extraction process was repeated with 50% ethanol (2×300 mL). The combined extracts were evaporated under reduced pressure to get 20-25% total dissolved solid (TDS). The concentrated solution was subjected to heating at temperature of about 95-100 °C for 6-8 hours under stirring. The solution was then kept for settling for 24 hours at room temperature. The lower layer was concentrated to dryness under vacuum at temperature of about 60-70 °C after the decantation of the upper layer. This provided the product as brown to dark brown color solid (1.0-1.2 g), withanolide glycosides: 1.33% comprises Withanoside IV: 0.68% & withanoside V/VI: 0.65%; total withanolide aglycones: 3.67% comprises Withanolide A: 1.89%, Withanolide B: 0.23% Withaferin A: 0.66% & 12-deoxywithastramonolide: 0.89% measured by HPLC. (United State Pharmacopeia—(USP) method with two markers).

### Example 2

By performing the same operation as provided in Example 1 with varied in the unit of operation can exert a variation in the result as follows:
withanolide glycosides: 6.43% comprises Withanoside IV: 3.14% & withanoside V/VI: 3.29%; total
withanolide aglycones: 12.08% comprises Withanolide A: 3.67%, Withanolide B: 0.90% Withaferin A: 5.21% & 12-deoxywithastramonolide: 2.30% measured by HPLC (USP method with two markers).

### Examples of Spray-Dried Powder Preparation

### Example 1

The Bioenhancer/emulsifier were weighed accurately and added to the purified water/appropriate solvent under stirring to prepare a solution. Ashwagandha extracts weighed accurately and added to the solution under stirring. The emulsifier and anticaking agent were added to the solution. The polymer was weighed precisely and added to the solution under continuous stirring. The final solution was spray dried to form a powder. Further, the spray-dried powder was shifted and packed.

| S.N. | Ingredients | Functional role | % w/w |
|---|---|---|---|
| 1 | Withania extract 6% | Active | 25.00 |
| 2 | Colloidal Silicon | Anticaking | 2.00 |
| | Dioxide (Aerosil) | agent | |
| 3 | Medium-Chain | Emulsifier | 6.00 |
| | Triglycerides (MCT) oil | | |
| | (RSPO)MB | | |
| 4 | Sunflower Lecithin | Bioenhancer/ | 1.00 |
| | (Phosphatidylcholine) | Emulsifier | |
| 5 | Hydroxypropyl Methyl | Polymer | 66.00 |
| | Cellulose E3 | | |
| | Total (Solid Content) | | 100.00 |

Note:
The Withania somnifera extract comprises in finished dose is 1.5%.

### Example 2

### Procedure: As Per Example 1

| S.N. | Ingredients | Functional role | % w/w |
|---|---|---|---|
| 1 | Withania extract 15% | Active | 54.00 |
| 2 | Colloidal Silicon | Anticaking | 2.00 |
| | Dioxide (Aerosil) | agent | |
| 3 | Medium-Chain | Emulsifier | 3.00 |
| | Triglycerides (MCT) oil | | |
| | (RSPO)MB | | |
| 4 | Sunflower Lecithin | Bioenhancer/ | 1.00 |
| | (Phosphatidylcholine) | Emulsifier | |
| 5 | Hydroxypropyl Methyl | Polymer | 40.00 |
| | Cellulose E3 | | |
| | Total (Solid Content) | | 100.00 |

Note:
The Withania somnifera extract comprises in finished dose is 8%.

### Example 3

The Bioenhancer/emulsifier was weighed accurately and added to the purified water/appropriate solvent under stirring to prepare a solution. Ashwagandha extracts were weighed accurately and added to the solution under stirring. The emulsifier and anticaking agent were added to the solution. The polymer was weighed precisely and added to the solution under continuous stirring. The final solution was spray-dried to form a powder. Further, the spray-dried powder was sifted and packed.

| S.N. | Ingredients | Functional role | % w/w |
|---|---|---|---|
| 1 | Withania somnifera | Active | 22.00 |
| | (Ashwagandha) extract 7% | | |
| 2 | Colloidal Silicon | Anticaking | 2.00 |
| | Dioxide (Aerosil) | agent | |
| 3 | Medium-Chain Triglycerides | Emulsifier | 6.00 |
| | (MCT) oil (RSPO)MB | | |
| 4 | Lecithin (Phosphatidylcholine) | Bioenhancer/ | 1.00 |
| | | Emulsifier | |
| 5 | Hydroxypropyl Methyl | Polymer | 69.00 |
| | Cellulose E3 | | |
| | Total | | 100.00 |

Note:
The Withania somnifera extract comprises a finished dose of 1.5%.

### Examples of Beadlets Preparation

### Example 1

The binder was weighed accurately and added to the purified water/appropriate solvent under stirring to prepare a solution. Ashwagandha extracts and diluent were weighed accurately and loaded in RMG container and dry mixed at appropriate impeller speed to form a uniform blend. The binder solution was added gradually to the dry blend in RMG to obtain wet mass. The wet mass was then passed through an extruder to obtain extrudes. Extrudes were loaded on a pitch spheronizer plate to obtain spherical beadlets/pellets. Spherical beadlets were loaded in a tray dryer for drying to obtain desired % LOD. Dried beadlets were sifted to obtain desired particle size distribution.

| S.N. | Ingredients | Functional role | % W/W |
|---|---|---|---|
| 1 | Withania somnifera extract 15% | Active | 10.00 |
| 2 | Hydroxypropyl Methyl | Binder | 3.00 |
| | Cellulose E3 | | |
| 3 | Microcrystalline Cellulose 101 | Diluent | 82.00 |
| 4 | Hydroxypropyl Methyl | Coating Agent | 5.00 |
| | Cellulose E3 | | |
| | Total (Solid Content) | | 100.00 |

Note:
The Withania somnifera extract comprises in finished dose is 1.5%.

### Example 2

### Procedure: As Per the Procedure Provided in Example 1

| S.N. | Ingredients | Functional role | % W/W |
|---|---|---|---|
| 1 | Withania somnifera extract 15% | Active | 54.00 |
| 2 | Hydroxypropyl Methyl | Binder | 3.00 |
| | Cellulose E3 | | |
| 3 | Microcrystalline Cellulose 101 | Diluent | 38.00 |
| 4 | Hydroxypropyl Methyl | Coating Agent | 5.00 |
| | Cellulose E3 | | |
| | Total (Solid Content) | | 100.00 |

Note:
The Withania somnifera extract comprises in finished dose is 8%.

### Example 3

The binder was weighed accurately and added to the purified water/appropriate solvent under stirring to prepare a solution. Ashwagandha extracts, anti-tacking agent and diluent were weighed accurately and loaded in RMG container and dry mixed at appropriate impeller speed to form a uniform blend. The binder solution was added gradually to the dry blend in RMG to obtain wet mass. The wet mass was then passed through an extruder to obtain extrudes. Extrudes were loaded on a pitch spheronizer plate to obtain spherical beadlets/pellets. Spherical beadlets were loaded in a tray dryer for drying to obtain desired % LOD. Dried beadlets were sifted to obtain desired particle size distribution. The coating agent was weighed accurately and added to the purified water/appropriate solvent under stirring to prepare a solution. Beadlets were coated with above coating solution.

| S.N. | Ingredients | Functional role | % W/W |
|---|---|---|---|
| 1 | Withania somnifera extract 7% | Active | 22.00% |
| 2 | Hydroxypropyl Methyl | Binder | 3.00 |
| | Cellulose E3 | | |
| 3 | Microcrystalline Cellulose 101 | Diluent | 70.00 |
| 4 | Hydroxypropyl Methyl | Coating Agent | 5.00 |
| | Cellulose E3 | | |
| | Total | | 100.00 |

Note:
The Withania somnifera extract comprises a finished dose of 1.5%.

### Examples of Oil Suspension Preparation

A Bioenhancer and emulsifiers were added in oils or mixture of oils under stirring and mixture was heated at 50-65 °C. Withania somnifera extract added to above mixture under stirring to form oil suspension.

| S.N. | Ingredients | Functional role | % w/w |
|---|---|---|---|
| 1 | Withania extract 7% | Active | 22.00 |
| 2 | Sunflower Oil | Carrier | 30.00 |
| 3 | Medium-Chain Triglycerides | Carrier | 45.00 |
| | (MCT) oil (RSPO)MB | | |
| 4 | Sunflower Lecithin | Bioenhancer/ | 3.00 |
| | (Phosphatidylcholine) | Emulsifier | |
| | Total | | 100.00 |

### Examples of Granules Preparation

The binder and Bioenhancer were weighed accurately and added to the purified water/appropriate solvent under stirring to prepare a solution. Ashwagandha extracts, anti-tacking agent, and diluent were weighed accurately loaded in an RMG container, and dried mixed at appropriate impeller speed to form a uniform blend. The binder solution was added gradually to the dry blend in RMG to obtain wet granules with optimum impeller speed. Wet granules were loaded in a tray dryer for drying to obtain desired % LOD. Dried granules were sifted to obtain desired particle size distribution

| | | Functional | |
|---|---|---|---|
| S.N. | Ingredients | role | % W/W |
| 1 | Withania somnifera extract 7% | Active | 22.00 |
| 2 | Hydroxypropyl Methyl | Binder | 3.00 |
| | Cellulose E3 | | |
| 3 | Colloidal Silicon | Anti-Tacking | 1.00 |
| | Dioxide (Aerosil) | Agent | |
| 4 | Microcrystalline Cellulose 101 | Diluent | 71.00 |
| 5 | Sunflower Lecithin | Bioenhancer/ | 3.00 |
| | (Phosphatidylcholine) | Emulsifier | |
| | Total | | 100.00 |

### Clinical Study

For the human pharmacokinetic studies as listed below, Withania somnifera compositions are prepared as per the examples as shown above.

### Study 1:

### Objectives of the Study:

To compare the bioavailability of total withanolides from a single oral dose of Product A—Withania somnifera composition 8%, Product B—Withania somnifera composition 1.5%, Product C—Reference 1 Withania somnifera composition 5% and Product D—Reference 2 Withania somnifera composition 10% in healthy adult human subjects under fasting conditions.
To compare the bioavailability of Withanoside IV, Withanolide A, 12-deoxy withanstramonolide, and Withaferin A from a single oral dose of—
   Product A—Withania somnifera composition 8%,
   Product B—Withania somnifera composition 1.5%,
   Product C—Reference 1 Withania somnifera composition 5% and
   Product D—Reference 2 Withania somnifera composition 10%; in healthy adult human subjects under fasting conditions.

### Study Design:

A Double Blind, Balanced, Randomized, Single-Dose, Four-Treatment, Four-Sequence, Four-Period, Cross-Over Oral Comparative Bioavailability Design in Healthy, Adult Human Subjects Under Fasting Conditions. There were four investigational products used in the study.
No. of subjects: 20 subjects

### Study Procedure:

The study was carried out in four periods. During the entire in-house stay from −24.00 hours prior to dosing till 24.00 hours post-dose, subjects were served dinner on the day of check-in of each period. Standard diet was served at around −23.00, −20.00, −16.00, −12.00 hours pre-dose. Thereafter, subjects were fasted for at least 10.00 hours prior to dosing and standard diet was served at 04.00, 08.00- and 12.00-hours post-dose, in each period. All meal plans were identical in all the periods of the study. In each period, drinking water was restricted to at least 01.00 hour prior to dosing until 01.00-hour post-dose (Except 240 mL of drinking water during dosing). After 01.00 hour, drinking water was provided ad libitum. Subjects remained in sitting position for the first 04.00 hours of post-dose and only necessary movements were allowed during this period. Subjects were not allowed to lie down (except as directed by the physician secondary to adverse events) during restriction period. Thereafter, subjects were allowed to move freely during the remaining part of the study. However, they were not allowed to take part in any kind of strenuous exercise/activity.

Subjects were instructed to abstain from consuming caffeine and/or xanthine products (i.e. coffee, tea, chocolate, and caffeine-containing sodas, colas, etc.), cigarettes and tobacco containing products for at least 24.00 hours prior to check-in of period-I till end of the study, alcohol and its products, grapefruit and its juice and opium and poppy containing foods for at least 48.00 hours prior to check-in of period-I till end of the study. Subjects were not allowed to take an unusual diet, for whatever reason (e.g. low salt) for 48.00 hours prior to check-in of period-I till end of the study. Subjects were selected based on abstinence from any prescription medications within 14 days prior to the study check and they were instructed not to take any prescription medications throughout the study. Subjects were also instructed not to take any over-the-counter medicinal products, or herbal medications throughout the study, and they were selected based on abstinence from over-the-counter medicinal products, and herbal medications, including Ashwagandha 07 days prior to check-in of period-I till the end of the study.

Compliance assessment to the above restrictions was performed prior to check-in of each period. A urine screen for drugs of abuse (Marijuana-THC, amphetamine-AMP, barbiturates-BAR, cocaine-COC, benzodiazepines-BZD, and morphine-MOR), and an alcohol test (by urine sample) was also performed on the day of check-in of each period.

Dose and Mode of Administration: In each period, after an overnight fasting of at least 10.00 hours, in the morning a single oral dose (two capsules) of Study Product (A or B or C or D) was administered with 240 mL of drinking water, (as per randomization schedule) at ambient temperature, to the subjects in sitting upright posture, under the supervision of Investigator or Medical officer and trained study personnel including the Quality Assurance auditor(s).
1. Product A—Withania somnifera composition 8% (50 mg providing 4 mg total withanolides)—Dose 2 capsules with each containing 25 mg providing 2 mg total withanolides as per the claimed composition method.
2. Product B—Withania somnifera composition 1.5% (125 mg providing 1.88 mg total withanolides)—Dose 2 capsules with each containing 62.5 mg providing 0.94 mg total withanolides as per the claimed composition method.
3. Product C—Reference 1 Withania somnifera composition 5% (600 mg providing 30 mg total withanolides)—Dose 2 capsules with each containing 300 mg providing 15 mg total withanolides.
4. Product D—Reference 2 Withania somnifera composition 10% (500 mg providing 50 mg Withanolide glycosides, 160 mg of Oligosaccharides, 52.5 mg of Withanolide aglycone (as Withaferin A)—Dose 2 capsules with each containing 250 mg providing 25 mg Withanolide glycosides & 80 mg Oligosaccharides, <1.25 mg Withanolide aglycone (as Withaferin A).

### Blood Sampling Time Points:

In each period, 13 blood samples were collected including the pre-dose sampling in each period. The pre-dose (00.00 hour) blood sample 06 mL was collected within 60 minutes prior to dosing. Post dose blood samples 06 mL were collected at 00.25, 00.50, 00.75, 01.00, 02.00, 03.00, 04.00, 05.00, 06.00, 09.00, 12.00 and 24.00 hours post dose respectively (+02 minutes window period).

All samples were collected in pre-labelled K2EDTA-Vacutainers from a forearm vein using an indwelling cannula as per the discretion of investigator. The Heparin-lock technique was used to prevent clotting of blood in the indwelling cannula. Before each blood sample was drawn via the indwelling cannula, 0.5 mL of blood was discarded so as to prevent the saline diluted blood and heparin (10 IU/mL) from interfering with the analysis.

After collection of blood samples, the collected samples were placed in a 25 hermos-insulated box containing wet ice and transferred the box to the sample processing room where the blood samples were centrifuged at 4000 RPM for 10 minutes at 4 °C to separate the plasma. Centrifugation was started within 30 minutes of the collection of samples, at each collection time-point. The resulting plasma was properly labelled, stored at −7015 °C until serum plasma analysis to evaluate pharmacokinetic parameters.

### Bioanalytical Procedure:

Total withanolides, Withanoside IV, Withanolide A, 12-deoxywithanstramonolide and Withaferin A were assayed in plasma using a validated Bioanalytical method.

Pharmacokinetic & Statistical parameters:

The estimated concentration vs. time profiles of Total withanolides, Withanoside IV, Withanolide A, 12-deoxywithanstramonolide, and Withaferin A were employed using a non-compartmental model of Phoenix WinNonlin® version 8.3.3 or higher version of Pharsight Corporation, USA, or SAS® system for windows version 9.4 or above (SAS® Institute Inc., USA) or application software for the following pharmacokinetic parameters.

Primary parameters: Cmax, AUC₀₋ₜ, and AUC_{0-∞}. Secondary parameters: Tmax, t_{1/2}, Kel.

Statistical analyses for Total withanolides were done using SAS® system for Windows version 9.4 or above (SAS® Institute Inc., USA) or applicable software. Total withanolides was calculated by adding data for Withanoside IV, Withanolide A, 12-deoxywithanstramonolide and Withaferin A.

Summary statistics, ANOVA, inter subject variability, 90% confidence intervals, and power were calculated for baseline corrected & baseline-uncorrected—Total withanolides pharmacokinetic data of Products A, B, C & D using SAS version 9.4 or higher version.

### Summary Statistics

Product A, Product B, Product C, Product D—Mean, standard deviation, standard error, minimum, median, maximum and coefficient of variation were calculated for plasma concentrations at each individual time point as well as for the pharmacokinetic parameters (AUC₀₋ₜ, AUC_{inf}, Cmax, Tmax, Kel, t_{1/2}) for baseline-corrected, baseline-uncorrected—Total withanolides. In addition, the following statistical information was provided for (AUC₀₋ₜ, AUC_{inf}, Cmax, Tmax, Kel, t_{1/2}) baseline—corrected, baseline—uncorrected Total withanolides:
i. Geometric LSM
ii. Ratio of Means
iii. 90% Confidence Intervals

### Results and Conclusion:

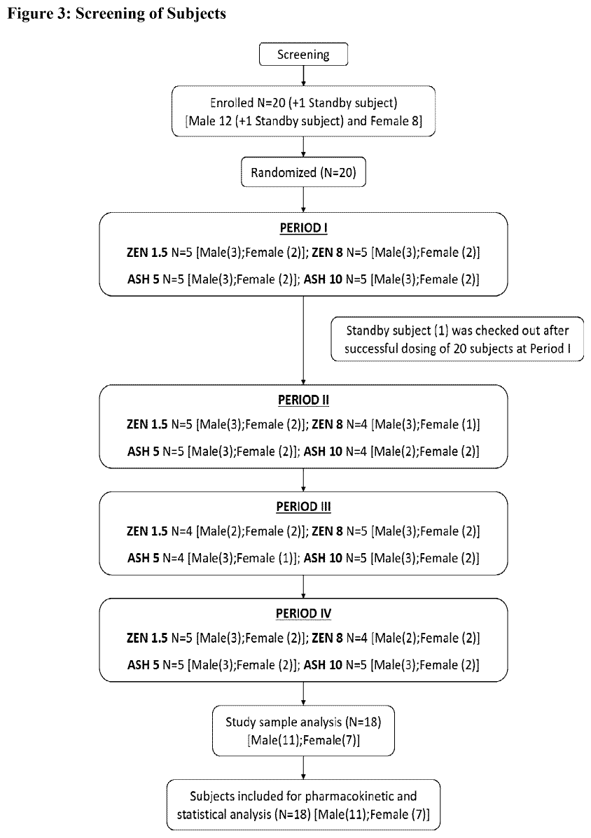

A total of 21 subjects were enrolled in this study while one subject out of 21 was considered as a standby subject. Twenty [Male (12) and female (8)] subjects were dosed in period-I. Eighteen [Male (11) and female (7)] subjects were dosed in period-II and III. Nineteen [Male (11) and female (8)] subjects were dosed in period-IV. Two subjects were dropped out from the study as these subjects did not report to the facility for period-II, III and IV. Out of these two subjects, one returned to the facility for period-IV. Hence, the subject was re-enrolled and dosed in period-IV. Totally eighteen [Male (11) and female (7)] subjects completed the four study periods. (FIG. 3).

A total of 20 subjects were enrolled into the study and their mean age, height, weight and BMI were 33 years, 163.03 cm, 66.55 kg and 25.05 kg/m² respectively (Table 1).

TABLE 1
Summary of subjects' demographics at baseline.

| | Age (years) | Gender [n (%)] | | BMI (kg/m²) |
|---|---|---|---|---|
| N | Mean ± SD | Female | Male | Mean ± SD |
| 20 | 33.55 ± 4.98 | 08 (40%) | 12 (60%) | 25.00 ± 2.73 |

N-Total subjects; n-total subjects in specified category; BMI-Body Mass Index; SD-Standard Deviation.

A. Product A Vs. Product C
Product A showed significantly greater bioavailability for Total Withanolides as compared to Product C based on significantly higher (P<0.05) Cmax & AUC parameters and their respective T/R ratio & 90% CI.
Product A showed—39 folds higher Cmax, 40.2 folds higher AUC0-t & 33.4 folds higher AUC0-inf as compared to Product C.
Tmax was similar whereas t½ was not significantly different between the groups.
   B. Product A Vs. Product D
Product A showed significantly greater bioavailability for Total Withanolides as compared to Product D based on significantly higher (P<0.05) Cmax & AUC parameters and their respective T/R ratio & 90% CI.
Product A showed—35.1 folds higher Cmax, 39.9 folds higher AUC0-t & 30.4 folds higher AUC0-inf as compared to Product D.
Product A took 33% longer time (Tmax) to reach the highest plasma concentration, and 33% faster clearance (t½) compared to Product D.
   C. Product B Vs. Product C
Product B showed significantly greater bioavailability for Total Withanolides as compared to Product C based on significantly higher (P<0.05) Cmax & AUC parameters and their respective T/R ratio & 90% CI.
Product B showed 38.5 folds higher Cmax, 33.9 folds higher AUC0-t & 27.2 folds higher AUC0-inf as compared to Product C.
Tmax was similar for both the groups, however, Product B showed 29% faster clearance (t½) compared to Product C.
   D. Product B Vs. Product D
Product B showed significantly greater bioavailability for Total Withanolides as compared to Product D based on significantly higher (P<0.05) Cmax and its respective T/R ratio & 90% CI.
AUC0-t and AUC0-inf were not significant for Product B compared to Product D based on p-value (dose normalized AUC0-t and AUC0-inf were significantly higher for Product B compared to Product D), however, 90% CI pointed towards supra-bioavailability.
Product B showed—36.9 folds higher Cmax, 34.8 folds higher AUC0-t & 26.7 folds higher AUC0-inf as compared to Product D.
Product B took 33% longer time (Tmax) to reach the highest plasma concentration, and 54% faster clearance (t½) compared to Product D.

### E. Total Withanolides Concentration Plasma and Bioavailability

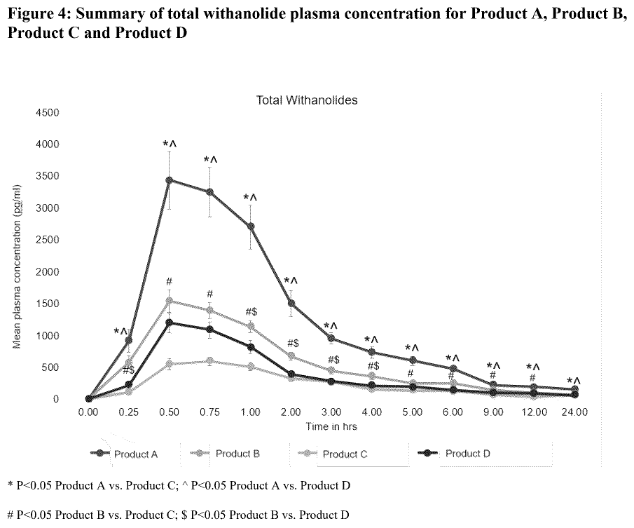

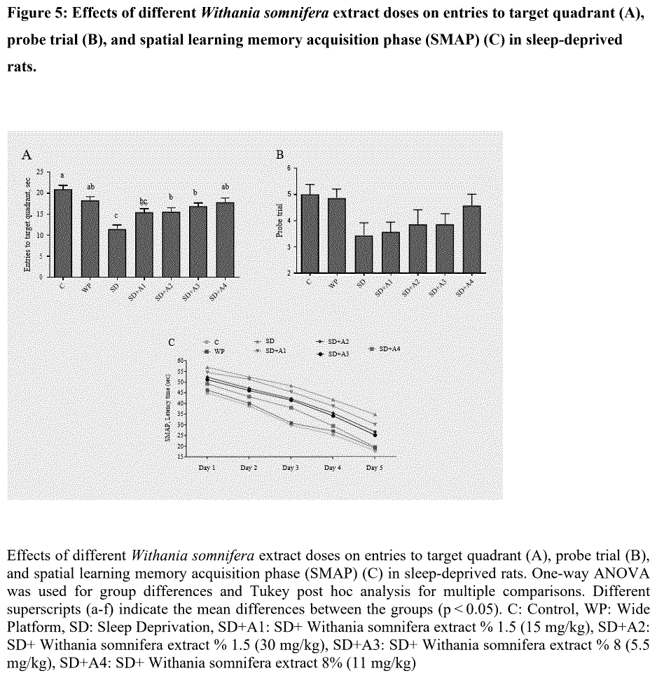

The summary of the total withanolides concentration in plasma post dose is shown in FIG. 4.
Product A showed significantly higher plasma total withanolides concentration at all time points post dose (0.25 to 24 hrs) as compared to Product C and Product D.
Product B showed significantly higher plasma total withanolides concentration at all postdose time points except 12 & 24 hrs as compared to Product C.
Product B showed a significantly higher plasma total withanolides concentration at 0.25, 1, 2, 3 & 4 hrs as compared to Product D.

TABLE 2
Summary results of total withanolides pharmacokinetic parameters
of Product A, Product B, Product C, and Product D.

| | | | T/R | | | |
|---|---|---|---|---|---|---|
| | Geometric Least Square Mean | | ratio | Relative | | |
| Parameters | Product A | Product C | (%) | Absorption | P - Value | 90% CI |
| Cmax | 3524 | 678 | 520 | 39.0 | <.0001* | 423.15-616.86 |
| AUC0-t | 10474 | 1956 | 535 | 40.2 | <.0001* | 384.03-685.98 |
| AUC0-inf | 12474 | 2804 | 445 | 33.4 | <.0001* | 278.35-611.65 |
| Tmax | 0.75 (0.50-1.00) | 0.75 (0.50-3.00) | — | — | 0.0808 | — |
| t½ | 7.98 ± 7.67 | 7.73 ± 6.56 | — | — | 0.9206 | — |
| | | | T/R | | | |
| | Geometric Least Square Mean | | ratio | Relative | | |
| Parameters | Product A | Product D | (%) | Absorption | P - Value | 90% CI |
| Cmax | 3542 | 1263 | 280 | 35.1 | <.0001* | 247.17-312.84 |
| AUC0-t | 10567 | 3310 | 319 | 39.9 | <.0001* | 245.08-392.92 |
| AUC0-inf | 12379 | 5086 | 243 | 30.4 | 0.0009* | 157.70-328.31 |
| Tmax | 0.75 (0.50-1.00) | 0.50 (0.50-12.00) | — | — | 0.7102 | — |
| t½ | 7.98 ± 7.67 | 11.97 ± 11.90 | — | — | 0.2909 | — |
| | | | T/R | | | |
| | Geometric Least Square Mean | | ratio | Relative | | |
| Parameters | Product B | Product C | (%) | Absorption | P - Value | 90% CI |
| Cmax | 1636 | 679 | 241 | 38.5 | <.0001* | 203.71-278.29 |
| AUC0-t | 4158 | 1959 | 212 | 33.9 | <.0001* | 174.55-249.46 |
| AUC0-inf | 4805 | 2817 | 171 | 27.2 | 0.0040* | 126.29-215.72 |
| Tmax | 0.75 (0.50-3.00) | 0.75 (0.50-3.00) | — | — | 1.0000 | — |
| t½ | 5.50 ± 3.79 | 7.73 ± 6.56 | — | — | 0.2223 | — |
| | | | T/R | | | |
| | Geometric Least Square Mean | | ratio | Relative | | |
| Parameters | Product B | Product D | (%) | Absorption | P - Value | 90% CI |
| Cmax | 1660 | 1198 | 139 | 36.9 | 0.0001* | 125.87-152.13 |
| AUC0-t | 4359 | 3336 | 131 | 34.8 | 0.0898 | 99.32-162.68 |
| AUC0-inf | 5086 | 5071 | 100 | 26.7 | 0.9910 | 59.25-140.76 |
| Tmax | 0.75 (0.50-3.00) | 0.50 (0.50-12.00) | — | — | 0.2942 | — |
| t½ | 5.50 ± 3.79 | 11.97 ± 11.90 | — | — | 0.0692 | — |

### Study 2:

### Objective:

In the study, the possible preventive effects of Withania somnifera extract on anxiety, stress, reduced cortisol and sleep induced by chronic sleep deprivation in rats were investigated. The Morris water maze (MWM) test was used to test spatial learning and memory. Potential molecular changes in the oxidative stress biomarkers, including the levels of cortisol, corticotropin-releasing hormone (CRH) and adrenocorticotropic hormone (ACTH), lipid peroxidation marker, MDA, and the activities antioxidant enzymes (GPx, CAT, SOD), levels of the brain neurotransmitters including serotonin (5-HT), noradrenaline (NE), and dopamine (DA), stress induced-expression of plasticity markers NCAM, ICAM-1, BDNF, NGF, GAP-43 and GABAergic receptors (GABAAR2, GABABR1, GABABR2) and 5-HT1A in the brain were also assessed.

### Material and Methods:

Animals—A total of 49 female Wistar rats (8-week-old) were used, and animals were reared at a temperature of (22±2 °C), humidity (55±5%), and a 12/12 h light/dark cycle.
Experimental Design—A total of 49 female Wistar rats (8 weeks) were randomly allocated into 7 groups (n=7 in each) as follows: C: Control, WP: Wide Platform, SD: Sleep Deprivation, SD+A1: SD+Withania somnifera extract % 1.5 (15 mg/kg), SD+A2: SD+Withania somnifera extract % 1.5 (30 mg/kg), SD+A3: SD+Withania somnifera extract % 8 (5.5 mg/kg), SD+A4: SD+Withania somnifera extract 8% (11 mg/kg).

The Ashwagandha and SD/(SD) groups were treated with (SD) at respective doses via oral gavage for 4 weeks. The control, SD, and WP groups were administered normal saline on a daily basis for 4 weeks via oral gavage. The SD and SD/Ashwagandha groups were sleep deprived (8 h/day, 8:30 am to 4:30 pm) for 4 weeks. All interventions including SD, Ashwagandha, and normal saline administration, started on the same day and continued for 4 weeks.

### Induction of Sleep Deprivation

Sleep deprivation was induced using multiple columns in water (modified platform) model (Alzoubi et al., 2016). The rats were placed in a large tank (170 cm in length×40 cm in width×55 cm in depth) made of glass filled with 24 °C water. The tank contained 20 small platforms with a diameter of 5 cm, placed 10 cm apart from edge to edge, and arranged in two rows. The tank was filled with water up to 2 cm below the platform surface. In this tank, animals (n=7 from the same group at each time) were capable of moving and jumping freely from one platform to another. When animals reached the paradoxical phase of sleep (Grahnstedt et al., 1985), muscle atonia set in and animals fell into the water and woke up, and they immediately climbed up to the platform and sat on it. The water in the tank was changed daily with a temperature of 24±1 °C. All rats had free access to food and water. To test the effect of possible stresses of the tank environment, WPs with a diameter of 12 cm were used to allow the rats to sleep without interruption or falling into water.

### Morris Water Maze

The Morris water maze (MWM) test was used to test spatial learning and memory among all groups of animals at the end of the study for 5 days. This model along with the detailed procedure was previously described (Sahin et al., 2011). At the end of the experiment, all rats were subjected to overnight fasting and blood was taken from decapitated animals via cervical dislocation. Blood samples were collected by gel biochemical tubes and serum samples were taken and centrifuged at 4 °C at 2370×g for 10 min in a chilled centrifuge. Brain samples were removed immediately from the skull and placed on a filter paper containing 0.2-M ice-cold sucrose solution, over a glass petri dish filled with crushed ice. Dissected hippocampus sections were placed in test tubes then immediately, stored in a deep freeze at −80 °C until analysis.

Based on FIG. 5, from day 1 to day 5, the distance travelled by sleep deprived rats were significantly decreased compared with normal controls rats which was significantly increased when treated with Withania somnifera extract in a dose dependent manner with highest improvement with 8% at higher dose. The time spent by rats supplemented with Withania somnifera extract to locate the hidden platform was significantly decreased compared to sleep deprived rats.

Result and Conclusion: The study results indicate that pretreatment with Withania somnifera extract ameliorated the long-term spatial memory.

### Biochemical Analysis

The concentration of serum biochemical parameters including lipid profile and liver, and renal functions was measured by ELISA (Elx-800, Bio-Tek Instruments Inc, Vermont, USA). The serum of MDA was analyzed by HPLC. Total antioxidant activity, antioxidant enzymes (SOD, CAT, GSHPx) was measured using the relevant kits.

### Result and Conclusion:

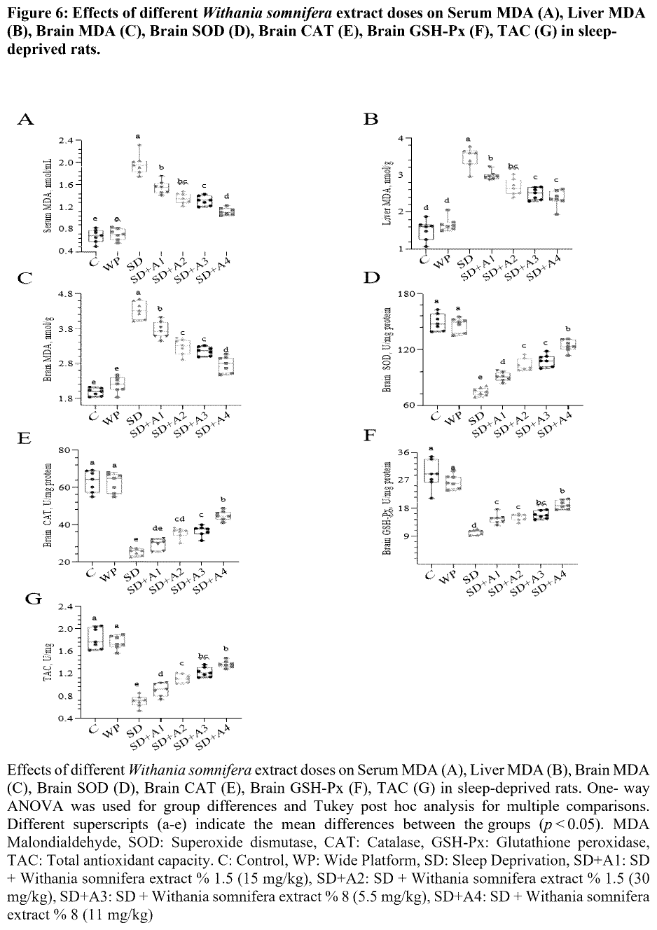

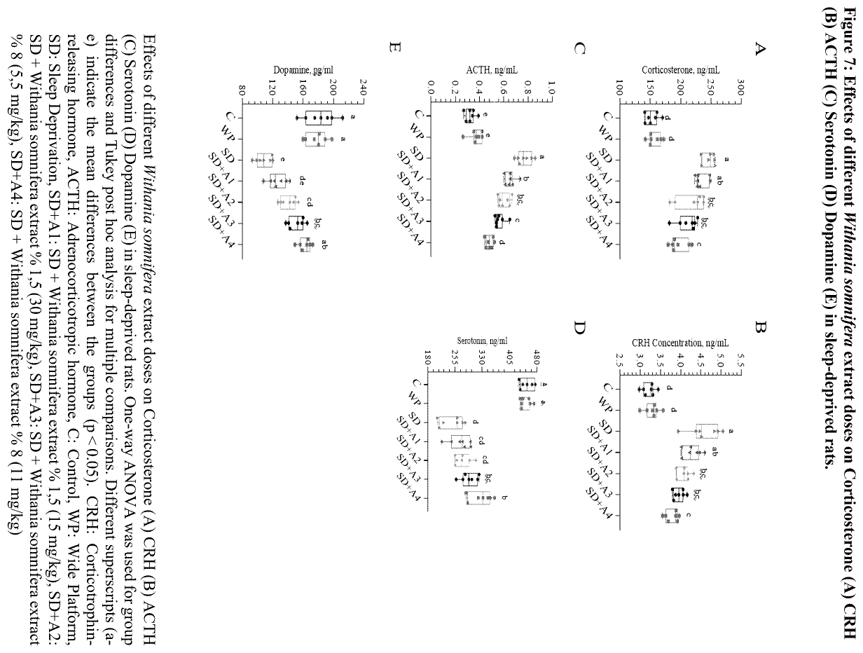

Based on FIG. 6, results indicate significant increase in serum as well as tissue MDA levels including brain tissue due to oxidative stress which was significantly reduced in mice administered Withania somnifera extract in a dose dependent manner for both 1.5 and 8% extract. Similarly, antioxidant systems in the body that include SOD, CAT and GSH-Px which were significantly reduced in case of sleep deprivation was subsequently restored in case of rats administered with Withania somnifera extract in a dose-dependent manner.

### Measurement of Stress Hormones

The measurement of stress hormones like Cortisol, corticotropin-releasing hormone (CRH) and ACTH was also measured by using ELISA. Levels of the brain neurotransmitters including serotonin (5-HT), noradrenaline (NE), and dopamine (DA) was also determined by ELISA.

Based on FIG. 7, Sleep deprivation significantly increased serum levels of stress hormones such as Corticosterone, CRH and ACTH which was significantly restored by Withania somnifera extract in a dose-dependent manner. Similarly, our results indicate that Withania somnifera extract helped to significantly restore serum dopamine and serotonin levels in sleep deprived rats.

### Western Blot Analysis

Brain plasticity markers NCAM, NCAM, BDNF, NGF, GAP-43 and GABAAR2, GABAB R1, GABAB-R2, 5HT1A was determined using the Western blot technique.

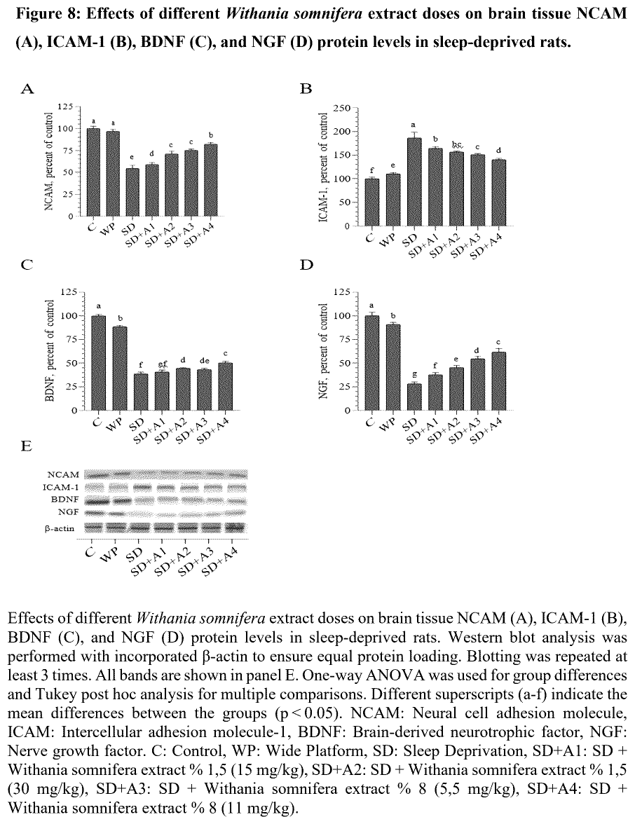

Based on FIG. 8, there was significant reduction in neuroprotective proteins such as NCAM, BDNF and NGF by sleep deprivation which was restored by Withania somnifera extract in a dose-dependent manner while inflammatory protein such as ICAM-1 levels which increased by sleep-deprivation was significantly reduced by Withania somnifera extract.

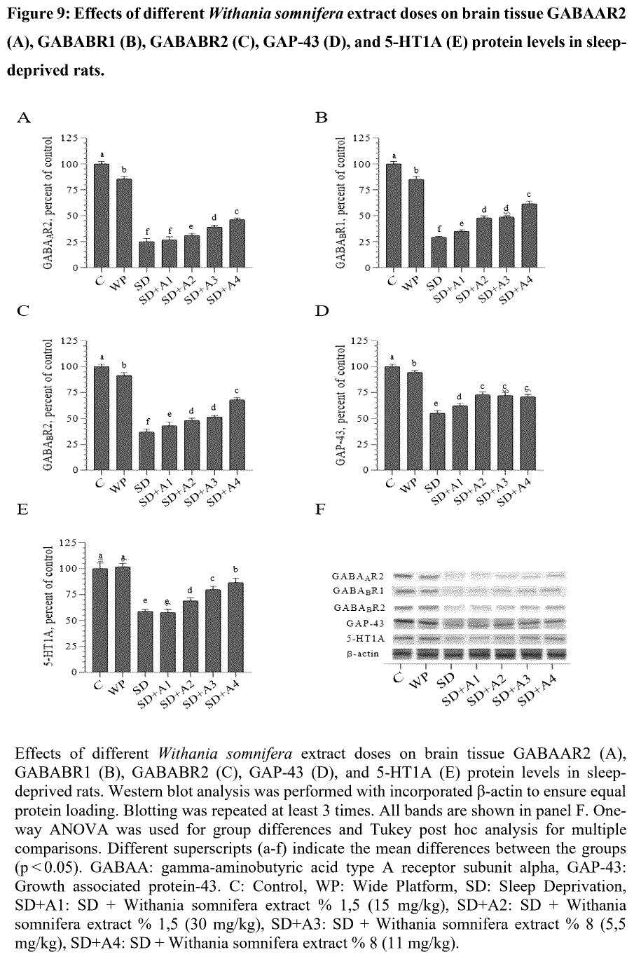

Based on FIG. 9, both Gamma-aminobutyric acid (GABA) and 5-HT1A receptors are expressed in almost all neuronal and glial cells and play a central role in modulation of spasticity, pain, addiction, anxiety or depression. Our results indicate that both 5-HT1A and GABA receptors levels are reduced during sleep deprivation which is reversed by Withania somnifera extract to a great extent. Brain tissue GAP43 is a nervous tissue-specific protein with neural outgrowth and regeneration and plays an important role in memory. We observed that sleep deprivation significantly reduced GAP43 proteins in brain tissue of rats which showed a reversing trend when supplemented with Withania somnifera extract.

1. An oral composition comprising Withania somnifera extract, wherein the total withanolides in the extract comprise withanolide glycosides and withanolide aglycones, in free or salt form.

2. The oral composition of claim 1, wherein the withanolide glycosides are selected from withanoside IV, withanoside V/VI, and combinations thereof.

3. The oral composition of claim 1, wherein the withanolide aglycones are selected from withanolide A, withanolide B, withaferin-A, 12-deoxywithastramonolide, and combinations thereof.

4. The oral composition of claim 1, wherein the extract comprises total withanolides in an amount of 1% to 40% w/w of the extract.

5. The oral composition of claim 1, wherein the extract comprises total withanolides in an amount of 15% to 40% w/w of the extract.

6. The oral composition of claim 1, wherein the withanolide glycosides are in an amount of 3% to 9.7% w/w of total withanolides.

7. The oral composition of claim 1, wherein the withanolide aglycones are in an amount of 8% to 16.6% w/w/ of total withanolides.

8. The oral composition of claim 2, wherein the withanolide glycosides are present in amounts (w/w total withanosides) selected from 0.54% to 1.94% of withanoside IV, 0.48% to 1.94% of withanoside V/VI, and combinations thereof.

9. The oral composition of claim 3, wherein the withanolide aglycones are present in amounts (w/w total withanosides) selected from 1% to 3.82% of withanolide A, 0.01% to 0.16% of withanolide B, 2.24% to 5% of withaferin-A, 0.96% to 2.33% of 12-deoxy withastramonolide, and combinations thereof.

10. The oral composition of claim 1, wherein the extract comprises total withanolides of 5% to 25% (w/w), wherein the total withanolides comprise
3% to 9.7% of withanolide glycosides (w/w total withanosides) selected from 0.54% to 1.94% of withanoside IV, 0.48% to 1.94% of withanoside V/VI and combinations thereof; and
8% to 16.6% of withanolide aglycones (w/w total withanosides) selected from 1% to 3.82% withanolide A, 0.01% to 0.16% of withanolide B, 2.24% to 5% of withaferin-A, 0.96% to 2.33% of 12-deoxy withastramonolide, and combinations thereof.

11. A method for prevention, improvement, and maintenance of cognitive health; improving level of stress; reducing cortisol; reducing brain-derived neurotropic factor (BDNF), managing anxiety; and/or improving sleep quality, comprising administering a composition according to claim 1 to a subject in need thereof.

12.-13. (canceled)

14. A process for preparing an extract of Withania somnifera comprising:
(i) obtaining a required material from one or more parts of the Withania somnifera plant;
(ii) obtaining an extract from the plant material by soaking it into an aqueous solvent and/or an organic solvent, or a combination thereof, for a suitable period and temperature to extract out the required constituents;
(iii) stirring the soaked plant material at a suitable temperature of about 50 °C to about 65 °C for a specified period;
(iv) optionally adjusting the suitable pH using a pH modifier, allowing it to settle at room temperature and filtering; followed by washing with water to remove the plant material; and
(v) drying and collecting the powder.

15. The process of claim 14, wherein the solvent is selected from an aqueous solvent is selected from the group consisting of water, aqueous phosphoric acid, aqueous hydrochloric acid and combination thereof; and/or an organic solvent is selected from the group consisting of methanol, ethanol, propanol, butanol, ethyl acetate, acetone, hexene, chloroform, and combination thereof.

16. (canceled)

17. The process of claim 14, wherein
the required material from one or more parts of the Withania somnifera plant of step (i) is Ashwagandha root;
the solvent of step (ii) is 50-55% ethanol solution; and
the period and temperature of step (ii) is about 3 hours at about 60 °C to about 65 °C.

18.-20. (canceled)

21. The oral composition of claim 1, further comprising one or more of pharmaceutically and/or nutraceutically acceptable excipients and/or carriers, e.g., selected from the group comprising solvents, bioavailability enhancers, PGP inhibitors, pH modifiers, emulsifiers, anticaking agents, coating agents and oils, cellulose derivatives, hydroxypropyl methyl cellulose, microcrystalline cellulose, polyacrylates, polyethylene glycols, povidones, starch and starch derivatives, gums, sugars, plant polysaccharides, and combinations thereof; e.g., wherein the oral composition is in a form selected from powders, granules, beadlets, caplets, tablets, capsules, soft gel capsules, solution, emulsions, and oil suspensions.

22.-23. (canceled)

24. A process for the preparation of oral Withania somnifera composition
A. in the form of powder wherein the process comprises:
(i) charging a solvent in a clean vessel;
(ii) adding bioenhancer, PGP inhibitor, pH modifier, and emulsifier under stirring to obtain a clear solution and heating the solution at temperature of about 50 °C to about 65 °C;
(iii) adding enriched Withania somnifera extract to the solution obtained in step (ii) under stirring and discontinuing the heating once a clear solution observes;
(iv) adding carrier to a solution, obtained in step (iii) under stirring; and
(v) spray drying the solution obtained from step (iv) to get powder; or
B. in the form of beadlets wherein the process comprises:
(i) preparing a solution of binder in a solvent;
(ii) adding enriched Withania somnifera extract composition, Anti-tacking agent and Diluent, and dry mix to form uniform blend
(iii) adding solutions prepared in step (i) in the dry blend of step (ii) to obtain the wet mass;
(iv) passing the wet mass through the extruder, followed by a spheronizer;
(v) drying and sifting the granules obtained in step (iv);
(vi) preparing coating solution using coating agent and solvent;
(vii) loading granules obtained in step (v) in fluidized bed coater and the solution obtained in step (vi); and
(viii) drying the granules obtained in step (vii); or
C. in the form of an oil suspension wherein the process comprises:
(i) adding bio-enhancers, PGP inhibitors, and emulsifiers in oils under stirring in a vessel and heating the mixture at temperature of about 50 °C to about 65 °C; and
(ii) adding enriched Withania somnifera extract to step (i) under stirring to obtain oil suspension; or
D. in the form of granules wherein the process comprises:
(i) preparing a solution of binder, and bioenhancer separately in a solvent;
(ii) adding enriched Withania somnifera extract, Anti-tacking agent and Diluent in mixer to form a uniform blend;
(iii) adding solutions prepared in step (i) in the dry blend of step (ii) to obtain the wet granules; and
(iv) drying and sifting the granules obtained in step (iii).

25.-27. (canceled)

28. The process of claim 24 wherein the solvent is selected from the group consisting of isopropyl alcohol, acetone, methanol, alcohol, ethyl acetate, ethanol, methylene dichloride, water, and combinations thereof; and/or
the bioenhancer is selected from the group consisting of piperine, d-limonene, monoglycerides, and phospholipids such as lecithin/phosphatidyl choline, glyceryl monosterate botanical extracts and combinations thereof; and/or
the PGP inhibitor is selected from the group consisting of piperine, Vitamin E TPGS (d-α-Tocopheryl polyethylene glycol 1000 succinate), alkaloids, polysorbates flavonoids, coumarins, resins, saponins and combinations thereof; and/or
the pH modifier is selected from the group consisting of sodium bicarbonate, citric acid, trisodium citrate, lactic acid, L-arginine, calcium carbonate, magnesium carbonate, and combinations thereof; and/or
the emulsifier is selected from the group consisting of stearic acid, phosphadylcholine, polysorbate, sugar alcohols, glycerol & derivatives and combination thereof; and/or
the polymer is selected from the group consisting of hydroxy propyl methyl cellulose, hydroxyl propyl cellulose, sodium carboxyl methyl cellulose, sodium alginates, carbomer, and combinations thereof; and/or
the anti-caking agent is selected from the group consisting of silicon dioxide, magnesium stearate, stearic acid, mannitol, and combinations thereof; and/or
the oil is selected from the group consisting of sunflower oil, coconut oil, corn oil, cottonseed oil, canola oil, olive oil, palm oil, peanut oil, safflower oil, sesame oil, soybean oil, rapeseed oil and medium-chain triglyceride (MCT) oil, and combinations thereof.

29.-35. (canceled)

36. The oral composition of claim 1 selected from
a. spray-dried powder comprising
25% of extract comprising 6% total withanolides,
2% of colloidal silicon dioxide,
6% of medium chain triglycerides,
1% of sunflower lecithin, and
66% hydroxypropyl methyl cellulose;
b. spray-dried powder comprising
54% of extract comprising 15% total withanolides,
2% of colloidal silicon dioxide,
3% of medium chain triglycerides,
1% of sunflower lecithin,
40% hydroxypropyl methyl cellulose;
c. spray-dried powder comprising
22% of extract comprising 7% total withanolides,
2% of colloidal silicon dioxide,
3% of medium chain triglycerides,
1% of sunflower lecithin,
69% hydroxypropyl methyl cellulose;
d. beadlets comprising
10% of extract comprising 15% total withanolides,
3% hydroxypropyl methyl cellulose binder,
82% microcrystalline cellulose,
5% hydroxypropyl methyl cellulose coating;
e. beadlets comprising
54% of extract comprising 15% total withanolides,
3% hydroxypropyl methyl cellulose binder,
38% microcrystalline cellulose,
5% hydroxypropyl methyl cellulose coating;
f. beadlets comprising
22% of extract comprising 7% total withanolides,
3% hydroxypropyl methyl cellulose binder,
70% microcrystalline cellulose,
5% hydroxypropyl methyl cellulose coating;
g. oil suspension comprising
22% of extract comprising 7% total withanolides,
30% sunflower oil;
45% medium chain triglycerides;
3% sunflower lecithin; and
h. granules comprising
22% of extract comprising 7% total withanolides,
3% hydroxypropyl methyl cellulose,
1% colloidal silicon dioxide,
5% sunflower lecithin;
wherein the percentages are by weight of the composition.

37. The oral composition of claim 36, wherein the total withanolides comprise 3% to 9.7% of withanolide glycosides (w/w total withanosides) selected from 0.54% to 1.94% of withanoside IV, and 0.48% to 1.94% of withanoside V/VI, and combinations thereof; and 8% to 16.6% of withanolide aglycones (w/w total withanosides) selected from of 1% to 3.82% withanolide A, 0.01% to 0.16% of withanolide B, 2.24% to 5% of withaferin-A, and 0.96% to 2.33% of 12-deoxy withastramonolide, and combinations thereof.

38. The oral composition of claim 1 in the form of 25 mg capsules providing 2 mg total withanolides, or 62.5 mg capsules providing 0.94 mg total withanolides.
